(19) United States
(12) Patent Application Publication
(10) Pub. No.: US 2017/0164727 A1
(43) Pub. Date: Jun. 15, 2017

(54) Oral Care Device

(71) Applicant: Jatin Vasant THAKKAR, Mumbai (IN)

(72) Inventor: Jatin Vasant THAKKAR, Mumbai (IN)

(21) Appl. No.: 15/320,015

(22) PCT Filed: Jun. 2, 2015
(86) PCT No.: PCT/IB2015/054167

§ 371 Date: Dec. 19, 2016

(30) Foreign Application Priority Data
Jun. 13, 2014 (IN) 1917/MUM/2014

(51) Int. Cl. A46B 15/00; A61B 17/24; A61C 15/04; A46B 9/04; A46B 5/00

(52) U.S. Cl. CPC A46B 15/0081; A46B 9/04; A46B 5/0037; A61C 15/046; A61B 17/244; A46B 2200/1066

(57) ABSTRACT

An oral care device is disclosed, which comprises a toothbrush having an elongated handle with bristles attached to an end thereof. The elongated handle includes a first portion and a second portion detachably connected with each other. Each of the first portion and the second portion has a cavity formed there-within. A resilient element, made of a resilient material, has a first edge disposed within the cavity of the first portion and a second edge disposed within the cavity of the second portion. The resilient element, in an in-operative configuration, is linearly disposed within the first portion and the second portion, thereby defining the operative configuration of the toothbrush. The resilient element in an operative configuration has at least a portion forming an arc, thereby defining an in-operative configuration of the toothbrush.

### FIELD OF DISCLOSURE

The present disclosure relates to an oral care device.

### BACKGROUND

Oral care is the practice of keeping the teeth, mouth and the tongue clean, to prevent dental problems such as cavities, decay, bad breath and the like. Oral care is also required for healing and regeneration of oral tissues. Toothbrushes, tongue cleaners and dental floss are some of the devices for maintaining oral hygiene. Toothbrushes are used to clean teeth and gums. Toothbrushes consist of an elongated handle having a head with tightly clustered bristles. Tongue cleaners are used to clean the bacterial build-up, food debris, fungi, dead cells and other foreign accumulations from the surface of the tongue. Dental floss is a thin filament used to remove food and dental plaque disposed between the teeth.

Conventionally, a toothbrush is suitable for cleaning the teeth, but it is not suitable for removing unpleasant coatings from the tongue and consequently falls short of accomplishing hygienic cleansing of the tongue, which may cause bad breath. Hence, conventional tongue cleaners are used to alleviate the above mentioned drawback. However, it is difficult to separately handle conventional toothbrushes and conventional tongue cleaners, especially during travel. Also, due to the flexible nature of conventional tongue cleaners, conventional tongue cleaners are prone to bend or even break during travel. Further, separately storing conventional toothbrushes and conventional tongue cleaners clutters the storage area and thereby aesthetically affects the storage area. Also, conventional tongue cleaners are available in pre-defined sizes and hence may cause difficulty for individuals to efficiently clean their tongue. Further, conventional tongue cleaners are susceptible to dirt from surrounding pollutants.

Accordingly, there is a need for an oral care device for reducing the above mentioned drawbacks.

### OBJECTS

Some of the objects of the system of the present disclosure which at least one embodiment herein satisfies are as follows:

It is an object of the present disclosure to ameliorate one or more problems of the prior art or to at least provide a useful alternative.

An object of the present disclosure is to provide an oral care device that has a toothbrush, a tongue cleaner and a dental floss.

Yet another object of the present disclosure is to provide an oral care device that is comparatively easy to handle.

Still another object of the present disclosure is to provide an oral care device that does not clutter storage areas thereby aesthetically enhancing the storage areas.

Yet another object of the present disclosure is to provide an oral care device that protects the tongue cleaner from the surrounding dirt and pollutants.

Still another object of the present disclosure is to provide an oral care device that protects the tongue cleaner and dental floss from bending and breaking.

An object of the present disclosure is to provide an oral care device wherein the size of the oral care device is adjustable as per the individual needs, thereby efficiently enabling tongue cleaning.

Other objects and advantages of the present disclosure will be more apparent from the following description when read in conjunction with the accompanying figures, which are not intended to limit the scope of the present disclosure.

### SUMMARY

An oral care device is disclosed in accordance with an embodiment of the present disclosure. The oral care device comprises a toothbrush having an elongated handle with bristles attached to an end thereof. The elongated handle includes a first portion and a second portion. The first portion and the second portion are detachably connected with each other. Each of the first portion and the second portion has a cavity formed there-within. A resilient element is nestled within the toothbrush. The resilient element has a first edge disposed within the cavity of the first portion and a second edge disposed within the cavity of the second portion, wherein the resilient element in an in-operative configuration is linearly disposed within the first portion and the second portion, by lockingly connecting the first portion and the second portion, and thereby defining operative configuration of the bristles of the toothbrush. The resilient element in an operative configuration has at least a portion forming an arc for cleaning the tongue and teeth, by spacing apart and angling the first portion and the second portion, thereby defining an in-operative configuration of the bristles of the toothbrush.

### BRIEF DESCRIPTION OF ACCOMPANYING DRAWINGS

The disclosure will now be explained in relation to the accompanying drawings, in which:

FIG. 1a illustrates a top view of an oral care device, in accordance with an embodiment of the present disclosure, wherein a first portion and a second portion are spaced apart from one another and a resilient element is slidably moveable in the first portion and the second portion;

FIG. 1b illustrates a side view of the oral care device of FIG. 1a;

FIG. 1c illustrates a side view of the oral care device of FIG. 1a wherein the first portion and the second portion are lockingly connected with each other;

FIG. 1d illustrates a side view of the oral care device of FIG. 1a wherein a resilient element is in an operative configuration;

FIG. 2a illustrates a top view of an oral care device, in accordance with another embodiment of the present disclosure, wherein a first portion and a second portion are spaced apart from one another and a resilient element is slidably moveable in the first portion and is fixedly connected in the second portion;

FIG. 2b illustrates a side view of the oral care device of FIG. 2a;

FIG. 2c illustrates a side view of the oral care device of FIG. 2a wherein the first portion and the second portion are lockingly connected with each other; and

FIG. 2d illustrates a side view of the oral care device of FIG. 2a wherein a resilient element is in an operative configuration.

### DETAILED DESCRIPTION

An oral care device of the present disclosure will now be described with reference to the embodiments which do not limit the scope and ambit of the disclosure. The description provided is purely by way of example and illustration.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The present disclosure illustrates an oral care device. The oral care device is used for maintaining oral care hygiene. The oral care device is used for cleaning the teeth, the gums and the tongue. Thus the oral care device enables preventing dental problems such as cavities, decay, bad breath, and the like. The oral care device is comparatively easy to handle and does not clutter storage areas thereby aesthetically enhancing the storage areas.

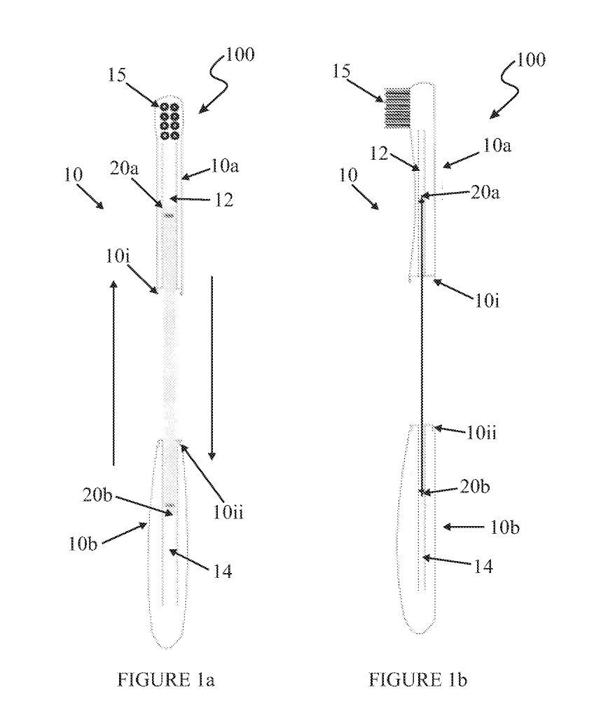

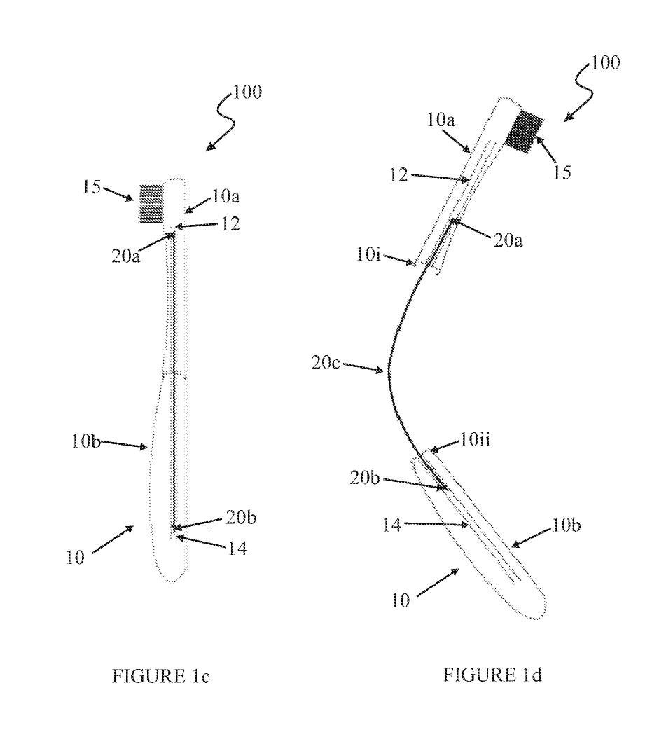

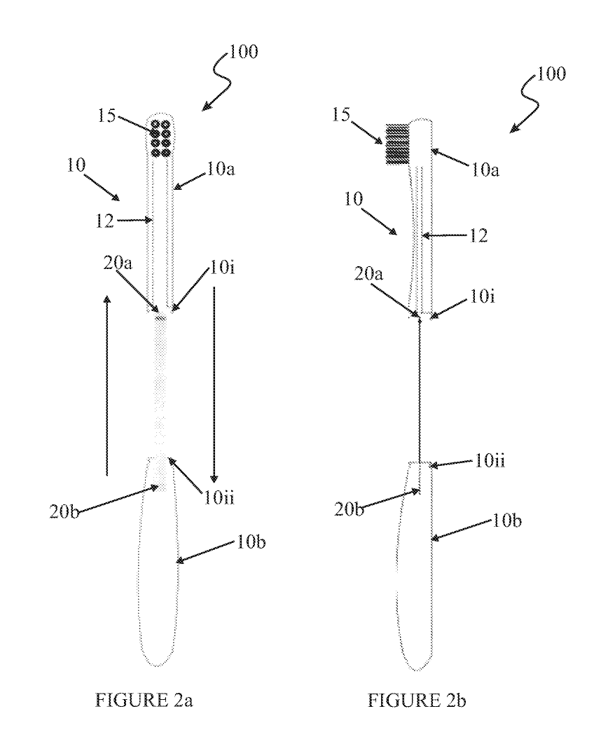

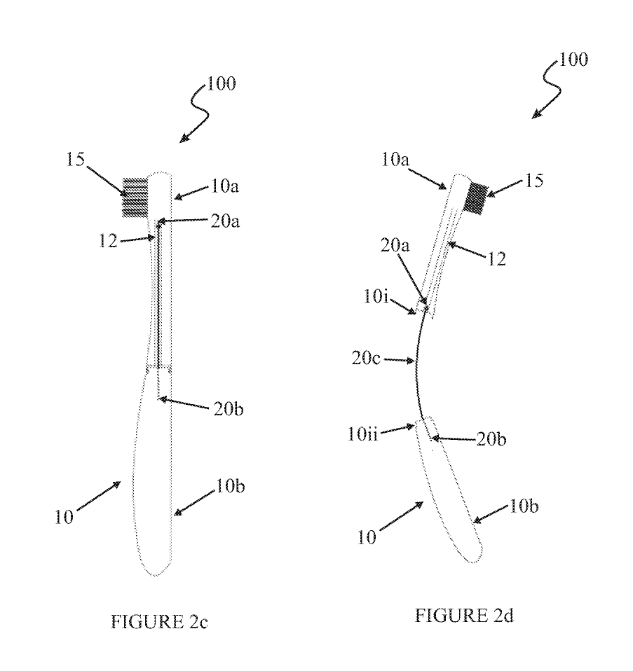

FIG. 1a and FIG. 2a of the accompanying drawings illustrate a side view of an oral care device 100 in accordance with an embodiment of the present disclosure. The oral care device includes a toothbrush and a resilient element nestlingly disposed within the toothbrush. The toothbrush includes an elongated handle 10 with bristles 15. The bristles 15 are attached to an end portion of the elongated handle 10. The elongated handle 10 includes a first portion 10a and a second portion 10b. The first portion 10a and the second portion 10b are detachably connected with each other. In one embodiment and as illustrated in FIG. 1a to FIG. 1d and FIG. 2a to FIG. 2d, the first portion 10a has a protrusion 10i adapted to be fitted on a slot 10ii configured in the second portion 10b. The protrusion 10i, fitted into the slot 10ii, defines a locking configuration of the first portion 10a with the second portion 10b. The protrusion 10i, detached from the slot 10ii, defines an un-locking configuration of the first portion 10a with the second portion 10b. In accordance with another embodiment, the protrusion 10i is disposed in the second portion 10b and the slot 10ii is configured on the first portion 10a.

Although, the locking and un-locking of the first portion 10a with the second portion 10b is carried out by using protrusion 10i and slot 10ii in this exemplary embodiment, it is not limited to this contrivance only, and any other locking and un-locking means may be used.

The first portion 10a has a cavity 12 formed there-within. The second portion 10b has a cavity 14 formed there-within. In one embodiment, the cavity 12 and the cavity 14 are coated with an antimicrobial coating. Typically, the antimicrobial coating is permanently added within the cavity 12 and the cavity 14. Alternatively, the antimicrobial coating is periodically added in the cavity 12 and the cavity 14.

The resilient element is made of a resilient material. The resilient element is nestled within the toothbrush. The resilient element has a first edge 20a and a second edge 20b. The first edge 20a is disposed within the cavity 12 of the first portion 10a and the second edge 20b is disposed within the cavity 14 of the second portion 20b.

In accordance with one embodiment and as illustrated in FIG. 1a to FIG. 1d, the first edge 20a and the second edge 20b are adapted to be nestled respectively into the first portion 10a and the second portion 10b.

In accordance with another embodiment and as illustrated in FIG. 2a to FIG. 2d, the first edge 20a is adapted to be nestled into the first portion 10a and the second edge 20b is fixedly connected within the second portion 10b.

In accordance with yet another embodiment, the first edge 20a is adapted to be fixedly connected within the first portion 10a and the second edge 20b is nestled into the second portion 10b.

In accordance with one embodiment of the present disclosure, at least one of the first portion 10a and the second portion 10b has at least one stopper (not illustrated in Figures). The stoppers prevent removal of at least one of the first edge 20a and the second edge 20b from the respective first portion 10a and the second portion 10b.

The resilient element, in an in-operative configuration, is linearly disposed within the first portion 10a and the second portion 10b by lockingly connecting the first portion 10a and the second portion 10b, thereby defining an operative configuration of the bristles 15 of the toothbrush. As the resilient element is disposed within the first portion 10a and the second portion 10b, the resilient element is not susceptible to dirt and other pollutants from the surrounding environment. Also, the first portion 10a and the second portion 10b form a protective cover for the resilient element thereby preventing bending or braking thereof, especially during travel.

The resilient element in an operative configuration, as illustrated in FIG. 1d and FIG. 2d, has at least a portion 20c forming an arc for cleaning the tongue or teeth. The arc is formed by spacing apart and angling the first portion 10a and the second portion 10b, thereby defining an in-operative configuration of the bristles 15 of the toothbrush.

More specifically, in an assembled configuration of the elongated handle 10, wherein the first portion 10a and the second portion 10b are lockingly connected with each other as illustrated in FIG. 1c and FIG. 2c, the oral care device 100 is used as a toothbrush for cleaning teeth and gums.

In a dis-assembled configuration of the elongated handle 10 the first portion 10a and the second portion 10b are spaced apart from each other as illustrated in FIG. 1a, FIG. 1b, FIG. 2a and FIG. 2b. The first portion 10a and the second portion 10b are then angled, as illustrated in FIG. 1d and FIG. 2d for forming an arc portion 20c for enabling cleaning of the tongue or the teeth with each portion 10a and 10b held in the hand of the user in the angled configuration, as illustrated in FIG. 1d and FIG. 2d.

In accordance with one embodiment of the present disclosure, the resilient element is used as a tongue cleaner. In accordance with another embodiment of the present disclosure, the resilient element is used as a dental floss.

As the oral care device 100 of the present disclosure is a single unit device formed by incorporating a resilient element that may be used as a tongue cleaner and a dental floss disposed within the toothbrush, hence the oral care device 100 is easy to handle, especially during travel. Also, as the oral care device 100 of the present disclosure is a single unit device the storage area of the oral care device 100 is clutter-free and hence is aesthetically appealing. Further, as the tongue cleaner of the oral care device 100 is manually adjustable as per the individual needs, the cleaning of the tongue by using the oral care device 100 is efficiently performed.

In accordance with one embodiment of the present disclosure, the toothbrush of the oral care device 100 may be powered by an external source such as a battery for cleaning the teeth and gums. In accordance with one embodiment of the present disclosure, the toothbrush of the oral care device 100 is moved for cleaning the teeth and gums.

### Technical Advancements

The device in accordance with the present disclosure has several technical advantages including but not limited to the realization of:
an oral care device that has a toothbrush and a resilient element, wherein the resilient element can be used as a tongue cleaner and a dental floss;
an oral care device that is comparatively easy to handle;
an oral care device that does not clutter storage areas thereby aesthetically enhancing the storage areas;
an oral care device that protects a resilient element from the surrounding dirt and pollutants
an oral care device that protects a tongue cleaner and a dental floss from bending and breaking; and
an oral care device wherein the size of the oral care device is adjustable as per the individual needs thereby efficiently enabling the tongue cleaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the invention to achieve one or more of the desired objects or results.

Any discussion of documents, acts, materials, devices, articles or the like that has been included in this specification is solely for the purpose of providing a context for the invention. It is not to be taken as an admission that any or all of these matters form a part of the prior art base or were common general knowledge in the field relevant to the invention as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the invention, unless there is a statement in the specification specific to the contrary.

While considerable emphasis has been placed herein on the specific features of the preferred embodiment, it will be appreciated that many additional features can be added and that many changes can be made in the preferred embodiment without departing from the principles of the invention. These and other changes in the preferred embodiment of the invention will be apparent to those skilled in the art from the disclosure herein, whereby it is to be distinctly understood that the foregoing descriptive matter is to be interpreted merely as illustrative of the invention and not as a limitation.

1. An oral care device comprising:
a toothbrush having an elongated handle with bristles attached to an end thereof, said elongated handle comprising a first portion and a second portion detachably connected with each other, each of said first portion and said second portion having cavities formed there-within; and
a resilient element nestled within said toothbrush, said resilient element having a first edge disposed within said cavity in said first portion and a second edge disposed within said cavity in said second portion,
wherein said resilient element
in an in-operative configuration is linearly disposed within said cavity in said first portion and said cavity in said second portion formed by lockingly connecting said first portion and said second portion thereby defining an operative configuration of said toothbrush,
in an operative configuration has at least a portion forming an arc by spacing apart and angling said first portion and said second portion thereby defining an operative configuration of said tongue cleaner.

2. The oral care device as claimed in claim 1, wherein said first edge is adapted to be fixed into said first portion and said second edge is adapted to be nestled into said second portion.

3. The oral care device as claimed in claim 1, wherein said second edge is adapted to be fixed into said second portion and said first edge is adapted to be nestled into said first portion.

4. The oral care device as claimed in claim 1, wherein said first edge and said second edge are adapted to be nestled into said first portion and said second portion respectively.

5. The oral care device as claimed in claim 1, wherein at least one of said first portion and said second portion has at least one stopper to prevent removal of at least one of said first edge and said second edge therefrom.

6. The oral care device as claimed in claim 1, wherein said first portion has a protrusion adapted to be fitted on a slot configured on said second portion for locking and un-locking said first portion with said second portion.

7. The oral care device as claimed in claim 1, wherein said second portion has a protrusion adapted to be fitted on a slot configured on said first portion for locking and un-locking said second portion with said first portion.

8. The oral care device as claimed in claim 1, wherein said cavities of said first portion and said second portion are coated with an antimicrobial coat.
